(19) United States
(12) Patent Application Publication
(10) Pub. No.: US 2024/0148718 A1
(43) Pub. Date: May 9, 2024

(54) INHIBITING MUTANT ISOCITRATE DEHYDROGENASE 1 (mIDH-1)

(71) Applicant: FORMA Therapeutics, Inc., Watertown, MA (US)

(72) Inventors: Susan ASHWELL, Carlisle, MA (US); Blythe THOMSON, Cincinnati, OH (US); Patrick F. KELLY, Concord, MA (US); Alan COLLIS, Lexington, MA (US); Jeff DAVIS, Hingham, MA (US); Duncan WALKER, Boulder, CO (US); Wei LU, Newton, MA (US)

(21) Appl. No.: 18/220,779

(22) Filed: Jul. 11, 2023

Related U.S. Application Data
(63) Continuation of application No. 17/243,177, filed on Apr. 28, 2021, now Pat. No. 11,738,018.
Application No. 17/243,177 is a continuation of application No. 16/431,588, filed on Jun. 4, 2019, now Pat. No. 11,013,733.
Application No. 16/431,588 is a continuation-in-part of application No. PCT/US19/32742, filed on May 16, 2019.
Application No. 16/431,588 is a continuation-in-part of application No. PCT/US19/32747, filed on May 16, 2019.
Application No. 16/431,588 is a continuation-in-part of application No. 16/414,505, filed on May 16, 2019, now Pat. No. 11,576,906.
Application No. 16/414,505 is a continuation-in-part of application No. 16/414,716, filed on May 16, 2019, now Pat. No. 10,532,047.
(60) Provisional application No. 62/692,591, filed on Jun. 29, 2018.
(60) Provisional application No. 62/672,462, filed on May 16, 2018.
(60) Provisional application No. 62/672,461, filed on May 16, 2018.
(60) Provisional application No. 62/812,367, filed on Mar. 1, 2019.
(60) Provisional application No. 62/798,677, filed on Jan. 30, 2019.
(60) Provisional application No. 62/798,681, filed on Jan. 30, 2019.
(60) Provisional application No. 62/798,684, filed on Jan. 30, 2019.
(60) Provisional application No. 62/798,687, filed on Jan. 30, 2019.
(60) Provisional application No. 62/798,690, filed on Jan. 30, 2019.
(60) Provisional application No. 62/773,562, filed on Nov. 30, 2018.
(60) Provisional application No. 62/692,598, filed on Jun. 29, 2018.
(60) Provisional application No. 62/692,601, filed on Jun. 29, 2018.
(60) Provisional application No. 62/692,604, filed on Jun. 29, 2018.
(60) Provisional application No. 62/692,605, filed on Jun. 29, 2018.
(60) Provisional application No. 62/680,566, filed on Jun. 4, 2018.
(60) Provisional application No. 62/680,571, filed on Jun. 4, 2018.
(60) Provisional application No. 62/680,560, filed on Jun. 4, 2018.
(60) Provisional application No. 62/680,562, filed on Jun. 4, 2018.
(60) Provisional application No. 62/812,367, filed on Mar. 1, 2019.
(60) Provisional application No. 62/798,677, filed on Jan. 30, 2019.
(60) Provisional application No. 62/798,681, filed on Jan. 30, 2019.
(60) Provisional application No. 62/798,684, filed on Jan. 30, 2019.
(60) Provisional application No. 62/798,687, filed on Jan. 30, 2019.
(60) Provisional application No. 62/798,690, filed on Jan. 30, 2019.
(60) Provisional application No. 62/773,562, filed on Nov. 30, 2018.
(60) Provisional application No. 62/692,591, filed on Jun. 29, 2018.
(60) Provisional application No. 62/692,598, filed on Jun. 29, 2018.
(60) Provisional application No. 62/692,601, filed on Jun. 29, 2018.
(60) Provisional application No. 62/692,605, filed on Jun. 29, 2018.
(60) Provisional application No. 62/680,566, filed on Jun. 4, 2018.
(60) Provisional application No. 62/680,571, filed on Jun. 4, 2018.
(60) Provisional application No. 62/680,560, filed on Jun. 4, 2018.
(60) Provisional application No. 62/680,562, filed on Jun. 4, 2018.
(60) Provisional application No. 62/672,462, filed on May 16, 2018.
(60) Provisional application No. 62/672,461, filed on May 16, 2018.
(60) Provisional application No. 62/692,591, filed on Jun. 29, 2018.
(60) Provisional application No. 62/672,462, filed on May 16, 2018.
(60) Provisional application No. 62/672,461, filed on May 16, 2018.
(60) Provisional application No. 62/712,160, filed on Jul. 30, 2018.
(60) Provisional application No. 62/701,487, filed on Jul. 20, 2018.

(51) Int. Cl. A61K 31/4709; A61K 31/706; A61P 35/00; A61P 35/02

(52) U.S. Cl. CPC A61K 31/4709; A61K 31/706; A61P 35/00; A61P 35/02

(57) ABSTRACT

Patients diagnosed with a cancer harboring an IDH-1 mutation can be treated by the administration of a therapeutically effective amount of a pharmaceutical composition comprising Compound 1, a selective inhibitor of 2-HG production from mIDH-1 enzymes including the R132 mutations R132C, R132H, R132L, R132G, and R132S.

### CROSS-REFERENCE TO RELATED APPLICATIONS

This application claims the benefit of and priority to U.S. Provisional Application No. 62/701,487, filed Jul. 20, 2018; and U.S. Provisional Application No. U.S. 62/712,160, filed Jul. 30, 2018; and

this application is a continuation-in-part of U.S. application Ser. No. 16/414,505, filed May 16, 2019, and International Application No. PCT/US19/32747, filed May 16, 2019, each of which claims the benefit of and priority to U.S. Provisional Application No. 62/672,461, filed May 16, 2018; U.S. Provisional Application No. 62/672,462, filed May 16, 2018; U.S. Provisional Application No. 62/680,566 filed Jun. 4, 2018; U.S. Provisional Application No. 62/680,571, filed Jun. 4, 2018; U.S. Provisional Application No. 62/680,560, filed Jun. 4, 2018; U.S. Provisional Application No. 62/680,562, filed Jun. 4, 2018; U.S. Provisional Application No. 62/692,598, filed Jun. 29, 2018; U.S. Provisional Application No. 62/692,601, filed Jun. 29, 2018; U.S. Provisional Application No. 62/692,604, filed Jun. 29, 2018; U.S. Provisional Application No. 62/692,605, filed Jun. 29, 2018; U.S. Provisional Application No. 62/692,591, filed Jun. 29, 2018, U.S. Provisional Application No. 62/773,562 filed Nov. 30, 2018; U.S. Provisional Application No. 62/798,677, filed Jan. 30, 2019; U.S. Provisional Application No. 62/798,681 filed Jan. 30, 2019; U.S. Provisional Application No. 62/798,684, filed Jan. 30, 2019; 62/798,687, filed Jan. 30, 2019; U.S. Provisional Application No. 62/798,690, filed Jan. 30, 2019; and U.S. Provisional Application No. 62/812,367, filed Mar. 1, 2019; and

this application is a continuation-in-part of U.S. application Ser. No. 16/414,716, filed May 16, 2019; and International Application No. PCT/US19/32742, filed May 16, 2019, each of which claims the benefit of and priority to U.S. Provisional Application No. 62/672,461, filed on May 16, 2018, U.S. Provisional Application No. 62/672,462, filed on May 16, 2018, and U.S. Provisional Application No. 62/692,591, filed on Jun. 29, 2018;

the contents of each of the applications listed above are hereby incorporated herein by reference in their entirety.

### TECHNICAL FIELD

The present disclosure relates to the treatment of cancer. In particular, the present disclosure provides methods of treating patients diagnosed with a cancer harboring certain mutant IDH-1 cancer cells.

### BACKGROUND

Dysregulation of metabolism is a common phenomenon in cancer cells. The NADP(+)-dependent isocitrate dehydrogenases 1 and 2 (IDH-1 and IDH-2) functionally modulate cellular metabolism in lipid synthesis, cellular defense against oxidative stress, oxidative respiration, and oxygen-sensing signal transduction. The presence of mutations in IDH-1 imparts a neomorphic activity to the enzyme, resulting in the production of (R)-2-hydroxyglutarate (2-HG), the downstream effects of which cause epigenetic changes that consequently block the proper differentiation of progenitor cells and lead to cancer. IDH-1 mutations have been reported in hematological malignancies, as well as many solid tumors types. By far the most frequent IDH-1 mutations occur at amino acid position R132, and include R132H, R132C, R132S, R132G, and R132L mutations.

Therapeutic compounds can be useful for inhibition of mutant IDH-1 and/or mutant IDH-2 cancer cells (mIDH-1 and mIDH-2) are being developed for the treatment of certain cancers. These therapies may also reduce elevated 2-HG levels in these cancer patients. Many different small molecule inhibitors of mutant isocitrate dehydrogenase (mIDH) proteins with neomorphic activity are disclosed in publications (e.g., WO2016/044789, WO2016/044787, WO2016/044782, WO2016/171755, and WO2016/171756), including testing of these compounds in IDH-1 R132H and IDH-1 R132C enzymatic assays, and cellular 2-HG assay using HCT116 mutant IDH-1 cells.

There remains a need for identifying therapeutic compounds that selectively inhibit the production of 2-HG from mIDH-1 cancer cells harboring R132 mutations including R132S, R132G and R132L. In addition, there remains a need for therapeutic compounds that selectively inhibit production of 2-HG from cancer cells harboring a variety of R132 IDH-1 mutations with clinically relevant comparative potencies, while remaining inactive at wild type IDH-1 cells. Preferably, a targeted, selective small molecule inhibitor of 2-HG production from mIDH-1 cancer cells is also inactive in mIDH-2 cancer cells that produce 2-HG. In addition, there is a need for inhibitors of the production of 2-HG from mIDH-1 cancer cells having a R132 mutation selected from the group consisting of: R132L, R132G, and R132S mutation in IDH-1.

### SUMMARY

The present disclosure provides methods for treating cancer. In particular, patients diagnosed with cancer harboring a mutant IDH-1 cancer cell, e.g., having a IDH-1 R132 mutation selected from the group consisting of: R132L, R132G, and R132S (in addition to R132H and R132C IDH-1 mutations), can be treated with a therapeutically effective amount of Compound 1. In some examples, patients treated with Compound 1 can have a mutant IDH-1 cancer that does not have a mIDH-2 mutation detected with a FDA approved mIDH-2 diagnostic (e.g., as provided at www.fda.gov/CompanionDiagnostics).

The patient can be diagnosed with a cancer (e.g., a hematologic malignancy such as MDS or AML) characterized by the presence of a mutant allele of IDH1 (e.g., a mIDH1 selected from the group consisting of: R132L, R132G, and R132S) and a concurrent mutation selected from the group consisting of FLT3, NPM1, CEBPA and TP53. Preferably, the cancer is not characterized by an IDH2 mutation. The patient can be treated with a therapeutically effective amount of Compound 1 (preferably, 150 mg of Compound 1 administered twice per day, each day) throughout a course of treatment (preferably, at least 6 months) as a single agent or in combination with another agent for treating the cancer (e.g., azacitidine).

Compound 1 is a small molecule inhibitor of mutated forms of isocitrate dehydrogenase 1 (IDH-1) enzyme. Compound 1 targets the mutant IDH-1 variants R132L, R132G, and R132S at lower concentrations than the wild-type IDH-1 enzyme or mutant IDH-2 enzymes tested in vitro as disclosed herein. Compound 1 is useful for the treatment of adult patients diagnosed with cancer having an IDH-1 mutation as detected by an FDA-approved test. Compound 1 can be administered to patients in need thereof in a therapeutically effective amount (e.g., 150 mg orally twice daily until disease progression or unacceptable toxicity). Patients for the treatment of cancer with Compound 1 can be selected based on the presence of IDH-1 mutations in the blood or bone marrow. In one embodiment, the recommended starting dose of Compound 1 is 150 mg taken orally twice daily with or without food until disease progression or unacceptable toxicity. For patients without disease progression or unacceptable toxicity, the patient can receive the therapeutically effective amount of Compound 1 for a minimum of 6 months to allow time for clinical response.

The invention is based in part on the discovery that Compound 1 selectively inhibits the production of 2-HG from mIDH-1 cancer cells harboring R132 mutations including R132S, R132G and R132L with clinically relevant comparative potencies, while remaining inactive at wild type IDH-1 cells. In addition, Applicants have discovered that Compound 1 is a targeted, selective small molecule inhibitor of 2-HG production from mIDH-1 cancer cells and is also inactive in mIDH-2 cancer cells that produce 2-HG (e.g., Compound 1 selectively inhibits the production of 2-HG from mIDH-1 cancer.

### BRIEF DESCRIPTION OF THE DRAWINGS

FIG. 1 illustrates Compound 1 binding with mIDH.

FIG. 2A and FIG. 2B are each a schematic of diaphorase-coupled assays used in Example 1, which measure activity by the determination of the level of remaining co-substrate NADPH after the enzymatic reaction is quenched.

FIG. 3A is a graph showing the results from a surface plasmon resonance (SPR) biophysical characterization of the molecular interaction between mIDH-1 inhibitor Compound 1 and recombinant IDH-1-R132H protein.

FIG. 3B is a comparator graph showing the SPR characterization of Compound 1 at a BCL6 control surface.

FIG. 4 is a synthetic reaction scheme for the preparation of Compound 1.

FIG. 5A illustrates the summary of cohorts from a phase 1 study in mIDH1 AML.

FIG. 5B illustrates use of Compound 1 in a phase 2 study in mIDH1 AML and MDS.

FIG. 6 is a graph showing strong correlation between ddPCR and NGS in AML patients from Example 7.

FIG. 7 is a graph showing good concordance in VAF between bone marrow analysis (BMA) and white blood cells (WB) in AML patients from Example 7.

FIG. 8 illustrates frequency of baseline co-mutations in AML patients from Example 7.

FIG. 9A and FIG. 9B illustrate change in IDH1 VAF across categories.

FIG. 10 is a graph showing that clinical response in a treatment naïve (TN) AML patient treated with Compound 1 in combination with azacitidine is associated with decrease in 2-HG and clearance of the IDH1m clone.

FIG. 11 is a graph showing that clinical response in a R/R AML patient treated with Compound 1 as a single agent is associated with decrease in 2-HG and clearance of the IDH1m clone.

FIG. 12 is a graph showing IDH2-mediated resistance.

FIG. 13 is a graph showing that presence of additional non-IDH1m clones drive resistance.

### DEFINITIONS

As used herein, the term “Course of Treatment” refers to the time period in which a patient is being administered an agent, including any administration holidays or recovery periods. A course of treatment can include a single treatment cycle or multiple treatment cycles. Additionally, a course of treatment can include a partial treatment cycle. The Course of Treatment can include the total time period during which a patient is on a treatment protocol for a disease, e.g. AML or MDS, with a therapy comprising the administration of a mIDH-1 inhibitor compound.

“Next-generation sequencing or NGS or NG sequencing” as used herein, refers to any sequencing method that determines the nucleotide sequence of either individual nucleic acid molecules (e.g., in single molecule sequencing) or clonally expanded proxies for individual nucleic acid molecules in a high-throughput fashion (e.g., greater than 103 or more molecules are sequenced simultaneously). Various next generation sequencing methods are known. In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, incorporated herein by reference. Next generation sequencing can detect a variant present in less than 5% of the nucleic acids in a sample. As shown in Example 7, for the purposes of Variant Allele Frequency analysis, digital droplet PCR (ddPCR) can also be used. ddPCR methods are known in the art, and are described in, e.g., Hindson B. J., et al. (2011). High-throughput droplet digital PCR system for absolute quantitation of DNA copy number. Anal. Chem. 83(22): 8604-8610, and Volegstein, B., et al. (1999) Digital PCR. Proc. Natl. Acad. Sci. USA 90: 9236-9241, incorporated herein by reference.

As used herein, the term “R132X mIDH-1 mutation(s)” refers to a mutation at the IDH-1 arginine 132 that results in inhibitory activity of Compound 1 against the mutated IDH-1 form harboring the R132 mutation. Preferably, the R132X mutations have a 2-HG IC50 value of less than 500 nM (most preferably less than 250 nM or less than 150 nM) using the in vitro assay of Example 1. Accordingly, preferred R132X mutations include R132H and R132C, as well as R132L, R132G, and R132S (or other R132X mutations having therapeutically relevant 2-HG IC50 values obtained using the in vitro assay of Example 1). Patients having R132X mIDH-1 mutation(s) can be identified using a suitable diagnostic, such as a diagnostic analyzing patient tissue with next generation sequencing technology that identified the presence of the R132X mIDH-1 mutation in the patient tissue sample.

As used herein, the term “R132X mIDH-1 Selective Inhibitor Therapy” refers to a therapy administered to a patient to inhibit the activity of R132X mIDH-1 in the patient, where the therapy is known to have selective inhibitory activity against R132X mIDH-1 over wild type IDH-1. An R132X mIDH-a selective inhibitor therapy can be administration of Compound 1 as disclosed herein.

As used herein, “sequencing” can be Next Generation Sequencing (NGS), a high-throughput sequencing technology that performs thousands or millions of sequencing reactions in parallel. Although the different NGS platforms use varying assay chemistries, they preferably generate sequence data from a large number of sequencing reactions run simultaneously on a large number of templates. The sequence data can be collected using a scanner, and then assembled and analyzed bioinformatically. Thus, the sequencing reactions are performed, read, assembled, and analyzed in parallel.

The terms “subject” and “patient” are used interchangeably in the present disclosure.

Susceptible IDH1 mutations are defined as those leading to increased levels of 2-hydroxyglutarate (2-HG) in the specified mIDH1 cancer cells (e.g., mIDH1 leukemia cells or mIDH1 glioma cells) and where efficacy is predicted by 1) clinically meaningful remissions with the recommended dose of Compound 1 and/or 2) inhibition of mutant IDH1 enzymatic activity at concentrations of Compound 1 sustainable at the recommended dosage according to validated methods. Susceptible mutations include R132H and R132C mIDH1 substitution mutations. In some methods, the susceptible IDH1 mutation leads to increased levels of 2-hydroxyglutarate (2-HG) in the leukemia cells. In some methods, efficacy of Compound 1 is predicted by a) clinically meaningful remissions with the recommended dose of Compound 1 and/or b) inhibition of mutant IDH1 enzymatic activity at concentrations of Compound 1 sustainable at the recommended dosage according to validated methods.

### DETAILED DESCRIPTION

Compound 1 is a small molecule mIDH-1 inhibitor useful for the treatment of patients harboring IDH-1 mutations, in both hematologic and solid tumors.

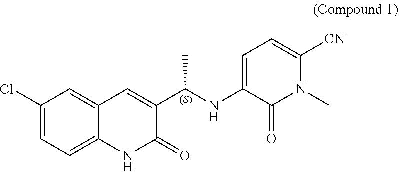

Compound 1 has potent and equivalent biochemical activity against a number of IDH-1 arginine 132 (R132) mutated forms, of which R132H and R132C are the most prevalent observed for human IDH-1. Compound 1 is a small molecule mIDH-1 (mutated isocitrate dehydrogenase 1) inhibitor. It is a permeable, orally bioavailable compound, with an excellent preclinical profile in both in vitro and in vivo models.

Isocitrate dehydrogenase (IDH) is a class of enzymes that catalyze the oxidative decarboxylation of isocitrate to α-keto-glutarate (α-KG). There are three isoforms in human cells. IDH-1 resides in the cytosol and peroxisomes, whereas IDH-2 and IDH-3 are mitochondrial enzymes. IDH-1 is dimeric and uses NADP+ as an electron acceptor. IDH-3 is a tetrameric enzyme and, in contrast, uses NAD+ as an electron acceptor. IDH-3 is the primary IDH enzyme participating in the Krebs cycle. The presence of the IDH-1 mutations imparts a neomorphic activity to the enzyme, resulting in the production of (R)-2-hydroxyglutarate (2-HG) which has been termed an “oncometabolite”, and has pleotropic roles in tumorgenesis.

Studies in genetically engineered mouse models and models derived from cancer patient samples both support the discovery that mIDH produces 2-HG, the downstream effects of which cause epigenetic changes that consequently block the proper differentiation of progenitor cells and lead to cancer. In particular, IDH-1 mutations can lead to the loss of wild type enzymatic activity (conversion of isocitrate to alpha-KG (α-KG)). Instead, the mutated enzymes acquire the neomorphic activity of converting α-KG to 2-HG. In mIDH-1 harboring cancer cells, wild type and mutant IDH-1 form a heterodimeric complex that can produce very high 2-HG levels. All IDH-1 mutations result in the formation of the (R)-enantiomer of 2-HG, which is contrast to the accumulation of (S)-enantiomer found in L2-HG aciduria patients, who harbor homozygous loss-of-function mutations in 2-HG dehydrogenase. Given the structural similarity between 2-HG and α-KG, 2-HG has been shown to be a competitive inhibitor of a number of α-KG dependent histone and DNA demethylases. 2-HG inhibits several KDM family histone demethylases in vitro, including H3K9/H3K36 demethylases KDM4A and KDM4C, and H3K36 demethylase KDM2A. Furthermore, elevated methylation levels of H3K4, H3K9, H3K27, and H3K79 have been observed in mIDH-1 containing patient-derived samples, as well as in cells expressing IDH mutations or treated with a cell-permeable ester of 2-HG. 2-HG also inhibits the TET family of DNA demethylases, which in turn results in the hypermethylation of DNA CpG islands. Mutations in IDH-1/2 and TET2 are thus far mutually exclusive, which supports the notion that 2-HG produced by mIDH inhibits TET2 and impairs hematopoietic cell differentiation. In addition, 2-HG has also been shown to block PHD activity, which is critical for regulation of hypoxia inducible factors and collagen hydroxylation and maturation. Hydroxylated collagen is important for the regulation of proliferation and proper differentiation of hematopoietic cells in bone marrow. Mutated IDH is also reported to block proper hepatocyte differentiation and promote cholangiocarcinoma. Since IDH-1 mutations are only found in tumor tissue, the present invention is based in part on the discovery of that the selective mIDH-1 inhibitor of Compound 1 can be developed as a targeted therapy for cancer. The patient selection biomarker for the use of Compound 1 can be the existence of IDH-1 mutation in a patient diagnosed with a cancer harboring mIDH-1.

Using in vitro cellular mechanistic assays monitoring levels of the errantly overproduced, tumorigenic metabolic byproduct 2-hydroxy glutarate (2-HG), inhibition of mIDH-1 results in a >90% reduction in levels of measured 2-HG, an effect that has also been shown to translate into similar levels of 2-HG suppression in in vivo PK-PD studies in HCT116 (IDH-1 R132H) and HCT116 (IDH-1 R132C) xenograft bearing mice. In both models, the free concentration of Compound 1 was comparable in plasma and xenograft tumors, and exposures were dose dependent. At the highest dose tested in these studies (50 mg/kg), Compound 1 inhibited 2-HG levels in tumor by >90% for up to 24 hours after the last dose in the HCT116 (IDH-1 R132H) xenograft model, and to similar levels for at least 12 hours in the HCT116 (IDH-1 R132C) model.

Accordingly, Compound 1 is useful in methods of treating patients diagnosed with a cancer harboring an IDH-1 mutation. The neomorphic enzymatic activity acquired as a result of IDH-1 mutation is believed to lead to the conversion of α-ketoglutarate (alpha-KG) to 2-hydroxyglutarate (2-HG). In consequence, patients bearing IDH-1 mutations have elevated levels of 2-HG. Most IDH-1 mutations result in a single amino acid change at the R132 residue, whereas most IDH-2 mutations occur at either Arginine 140 (R140) or Arginine 172 (R172). The IDH mutation spectrum varies among different tumor types (Table 1).

TABLE 1

| | Total Mutation | |
|---|---|---|
| Tumor Types | Frequency | IDH Mutation Identities |
| Glioma | 70-90% | IDH1^{R132H}, IDH1^{R132C}, IDH1^{R132S}, IDH2^{R172K} |
| AML | 10-30% | IDH2^{R140Q}, IDH1^{R132H}, IDH1^{R132C}, IDH2^{R172K}, |
| | | IDH1^{R132G}, IDH1^{R132S} |
| Chondrosarcoma | 75% | IDH1^{R132C}, IDH1^{R132H} |
| Intrahepatic | 10-25% | IDH1^{R132C}, IDH1^{R132L}, IDH1^{R132G}, IDH1^{R132H}, |
| Cholangiocarcinoma | | IDH2^{R172W} |

For example, IDH-1 R132 mutations represent more than 90% of the IDH mutations present in low grade glioma and secondary GBM patients. IDH-1 mutations have been reported in hematological malignancies such as acute myeloid leukemia (AML) and myelodysplastic syndrome (MDS), as well as many solid tumors types, including low grade glioma, secondary glioblastoma, intrahepatic cholangiocarcinoma (IHCC), chondrosarcoma, and melanoma. By far the most frequent IDH-1 mutations occur at amino acid position R132, and include R132H, R132C, R132S, R132G, and R132L mutations. Given that Compound 1 is a potent inhibitor of a spectrum of different IDH-1 R132 mutations, but is inactive against either wild type IDH-1 or mutated IDH-2, patients will be selected based on the occurrence of an IDH-1 mutation at the R132 residue.

The patient can be diagnosed as having an IDH-1 R132 mutation disclosed herein using sequencing methods, such as next-generation sequencing methods. The diagnostic patient selection method can be a next-generation sequencing (NGS)-based tumor genotyping assay analyzing a patient tissue sample such as a bone marrow sample. Useful techniques and technologies for diagnosing a patient as having a IDH-1 R132 mutation may include, without limitation, sequencing machines and/or strategies well known in the art, such as those developed by Illumina/Solexa (the Genome Analyzer; Bennett et al. (2005) Pharmacogenomics, 6:373-20 382), by Applied Biosystems, Inc. (the SOLiD Sequencer; solid.appliedbiosystems.com), by Roche (e.g., the 454 GS FLX sequencer; Margulies et al. (2005) Nature, 437:376-380), and by others.

In some methods, a therapeutically effective amount of Compound 1 can be administered to a patient having a mIDH1 mutation at R-132 and a concurrent mutation at FLT3. The FMS-like tyrosine kinase 3 (FLT3) gene encodes a membrane bound receptor tyrosine kinase that affects hematopoiesis leading to hematological disorders and malignancies. FLT3 is one of the frequently mutated genes in hematological malignancies, such as adult acute myeloid leukemias (AML). The presence of a FLT3 internal tandem duplication has been detected in patients with acute myeloid leukemia (AML) and patients diagnosed with intermediate and high risk myelodysplastic syndrome (MDS). The heightened frequency of constitutively activated mutant FLT3 in adult AML has made the FLT3 gene a highly attractive drug target in this tumor type. A method for treating a FLT3 mutated mIDH1 proliferative disorder can comprise identifying a mIDH1 R132 mutation in a patient and measuring expression of a mutated FLT3 or a constitutively active FLT3 mutant, and one or more genetic abnormalities in a sample obtained from a tumor sample obtained from the patient; and administering to the patient a therapeutically effective amount of Compound 1 or a pharmaceutically acceptable salt thereof (e.g., 150 mg Compound 1 BID) for 6 months or more. Useful techniques and technologies for diagnosing a patient as having a IDH-1 R132 mutation may include, without limitation, sequencing machines and/or strategies well known in the art, such as those developed by Novartis (e.g. LeukoStrat® CDx FLT3 (https://www.accessdata.fda.gov/cdrh_docs/pdf16/p160040c.pdf)).

A method of treating a patient with acute myeloid leukemia (AML), can comprise: (a) analyzing a genetic sample isolated from the patient for the presence of cytogenetic abnormalities and a mutation in at least one of FLT3, NPM1, CEBPA, IDH1, and TP53 genes; and (b) treating the patient by administering a therapeutically effective amount of Compound 1 to the patient (e.g., a total of 150 mg of Compound 1 BID each day) if the mutation is present in R132 mIDH1 and at least one of FLT3, NPM1, CEBPA and TP53 genes.

Compound 1 can be administered as a single agent as the R132X mIDH-1 Selective Inhibitor Therapy, or in combination with other therapeutic agents that are not mIDH-1 inhibitors as a combination for the R132X mIDH-1 Selective Inhibitor Therapy. As used herein, the term “R132X mIDH-1 mutation(s)” refers to a mutation at the IDH-1 arginine 132 that results in inhibitory activity of Compound 1 against the mutated IDH-1 form harboring the R132 mutation.

In some methods, Compound 1 is administered to a patient diagnosed as having a R132 IDH1 mutation either as a single agent or in combination with azacitidine. In some examples, patients have been treated with or are already being treated with azacitidine. In some embodiments, a combination therapy of Compound 1 and azacitidine can be administered for the treatment of patients with a cancer harboring aIDH-1 mutation (e.g., mIDH1 forms of AML). For example, patients can be administered Compound 1 daily (BID) in continuous 28-day cycles, in combination with azacitidine (administered at the dose of 75 mg/m² for 7 days IV/SC per every 28-day cycle).

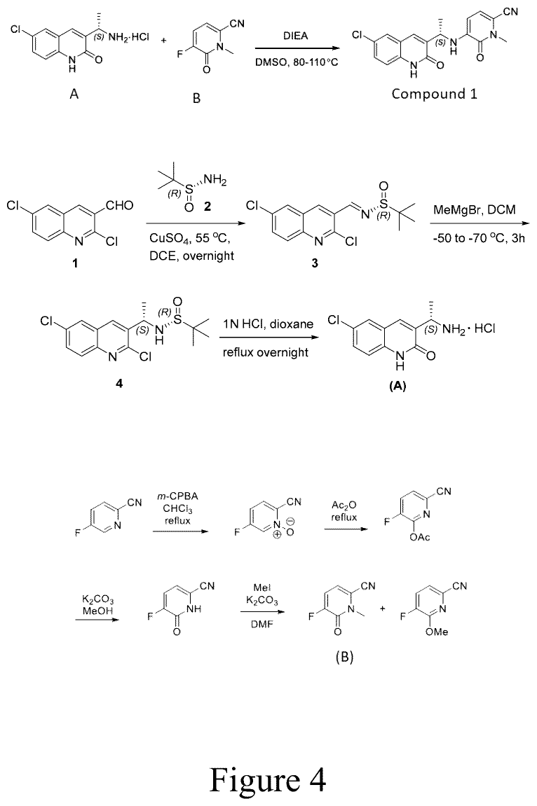

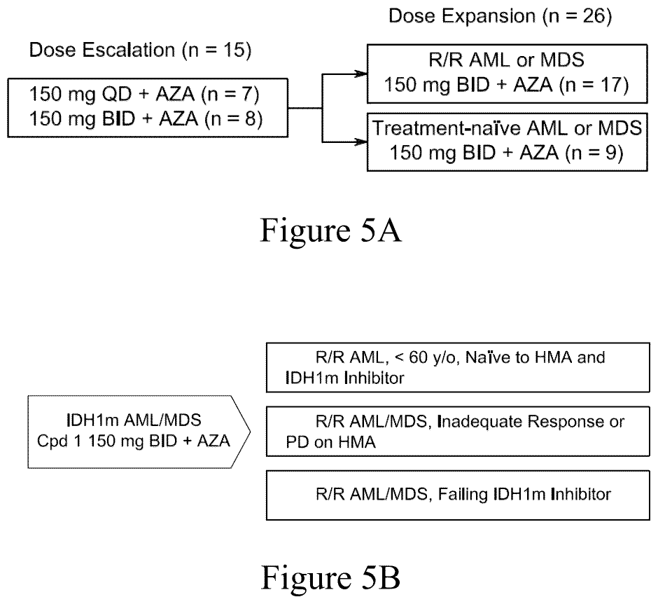

Referring to FIG. 4, Compound 1 can be prepared in a convergent synthesis from Intermediate A and Intermediate B as shown in FIG. 4 via the nucleophilic displacement reaction under basic conditions of (S)-3-(1-aminoethyl)-6-chloroquinolin-2(1H)-one (Intermediate A) and the fluoropyridone (Intermediate B). 1H, 13C NMR and mass spectral data are consistent with the assigned structure. The asymmetric synthesis of Intermediate A started with the condensation of the commercially available quinoline aldehyde (1) with (R)-tert-butanesulfinamide (2) to form the chiral (R)-N-tert-butanesulfinimine (3), followed by addition of methyl magnesium bromide in dichloromethane to yield the desired product (4) as the major diastereoisomer (dr: 98:2). Cleavage of the chiral auxiliary and simultaneous hydrolysis of 2-chloroquinoline moiety under mildly acidic conditions using 1N HCl in dioxane gave Intermediate A in quantitative yield. The structure of Intermediate A was confirmed by NMR and mass spectroscopy, and the enantiomeric purity was determined by chiral SFC analysis. The (S)-stereochemistry was confirmed by X-ray co-crystal structures of several inhibitor analogs prepared from the same chiral amine intermediate bound to mIDH-1 R132H. Intermediate (B) was prepared from commercially available 5-fluoropicolinonitrile in four steps. N-oxidation of 5-fluoropicolinonitrile followed by reflux of the N-oxide in acetic anhydride gave acetate, following work-up and purification. Solvolysis of the acetate group followed by N-methylation under standard conditions gave a mixture of N-methylated and O-methylated products (4:1). The minor O-methylated product was removed by column chromatography. NMR and mass spectral data are consistent with the structure of Intermediate Compound (B). Compound 1 (5-{[(1S)-1-(6-chloro-2-oxo-1,2-dihydroquinolin-3-yl)ethyl]amino}-1-methyl-6-oxo-1,6-dihydropyridine-2-carbonitrile) has a molecular weight of 355 with a melting point onset temperature of 251.3 C (DSC) and peak maximum 254.1 ° C.

The present disclosure also contemplates, among other things, the following numbered embodiments:
1. A method of treating a patient diagnosed with a cancer harboring a cancer cell with an IDH-1 R132 mutation selected from the group consisting of: R132L, R132G, and R132S, the method comprising administering to the patient in need thereof a therapeutically effective amount of Compound 1.
2. The method of embodiment 1, wherein the cancer does not harbor a IDH-2 mutation.
3. The method of embodiment 1, wherein the cancer does not harbor a IDH-2 mutation selected from the group consisting of: IDH-2 R172K and IDH-2 R140Q.
4. The method of embodiment 1, wherein the patient is diagnosed as having a R132 mutation based on a patient diagnostic.
5. The method of embodiment 4, wherein the patient diagnostic comprises detecting the R132 mutation in a tissue sample obtained from the patient.
6. The method of embodiment 5, wherein the tissue sample is obtained from the bone marrow of the patient.
7. The method of any one of embodiments 4-6, wherein the R132 mutation is detected using next generation sequencing (NGS) without the use of PCR.
8. A method of treatment comprising the steps of:
   a. selecting a patient for treatment based on the presence of one or more IDH-1 mutations selected from the group consisting of: R132L, R132G, and R132S;
   b. administering Compound 1 to the selected patient from step (a) at a starting dose of 150 mg taken orally twice daily until disease progression or unacceptable toxicity.
9. The method of embodiment 8, where the IDH-1 mutation is detected in cancer cells obtained from the blood or bone marrow of the patient.
10. The method of embodiment 9, wherein the IDH-1 mutation is detected prior to administering Compound 1 to the patient.
11. The method of any one of embodiments 1-10, comprising the step of detecting the IDH-1 mutation in a cell from the patient using a next-generation sequencing (NGS)-based tumor genotyping assay.
12. The method of any one of embodiments 1-11, wherein administration of Compound 1 to the patient results in a decreased 2-hydroxyglutarate (2-HG) levels in the blood of the patient within the first 15 consecutive days of treatment of the patient with Compound 1.
13. The method of any one of embodiments 1-12, wherein the method comprises administering 150 mg of Compound 1 to the patient in the solid form obtained from the method of Example 5.
14. The method of any one of embodiments 1-12, wherein the method comprises administering 150 mg of Compound 1 to the patient twice daily throughout a course of treatment.
15. The method of embodiment 14, wherein the course of treatment is at least 15 consecutive days.
16. The method of any one of embodiments 1-15, wherein Compound 1 is administered to the patient once every 12 hours on consecutive days throughout a course of treatment.
17. The method of any one of embodiments 1-16, wherein Compound 1 is administered to the patient throughout a course of treatment of at least 6 months.
18. A method of inhibiting the production of 2-HG from a cell harboring a IHD-1 mutation selected from the group consisting of: R132L, R132G and R132S, the method comprising contacting the cell with Compound 1 in an amount, under conditions, and for a time sufficient to inhibit the production of 2-HG from the cell.
19. A method of treating a patient diagnosed with a cancer harboring a cancer cell with an IDH-1 R132 mutation, the method comprising administering to the patient in need thereof a therapeutically effective amount of Compound 1.
20. The method of embodiment 19, wherein the patient is diagnosed with a cancer harboring an IDH-1 R132 mutation in a cell obtained from the patient, prior to the administration of Compound 1.
21. A method of treating a patient diagnosed with a cancer harboring a cancer cell with an IDH-1 R132 mutation, the method comprising administering to the patient in need thereof a therapeutically effective amount of Compound 1.
22. The method of embodiment 21, wherein the patient is diagnosed with a cancer harboring an IDH-1 R132 mutation in a cell obtained from the patient, prior to the administration of Compound 1.
23. A method of treating a patient diagnosed with a cancer, the method comprising
   a. diagnosing the patient as having a mutant IDH-1 mutation in a cell obtained from the patient; and
   b. administering a therapeutically effective amount of a pharmaceutical composition comprising Compound 1 to the patient in need of an inhibitor of the mutant IDH-1 enzyme that targets the mutant IDH-1 variants R132C at no greater than about 5 times the level of R132H; and
   c. continuing to administer the pharmaceutical composition to the patient throughout a course of treatment of at least 6 months.
24. The method of embodiment 23, wherein the patient is in need of an inhibitor of mIDH-1 variants selected from the group consisting of R132L, R132G, and R132S;
25. The method of any one of embodiments 23-24, wherein the relative targeting of R132C and R132H variants of mIDH-1 is measured by the ratio of IC₅₀ values obtained using the assay of Example 3.
26. The method of any one of embodiments 23-25, wherein the patient is diagnosed as having an IDH-1 mutation in a cell from the patient using a next-generation sequencing (NGS)-based tumor genotyping assay.
27. The method of any one of embodiments 23-26, wherein the pharmaceutical composition is administered to the patient twice per day.
28. The method of any one of embodiments 23-27, wherein the pharmaceutical composition is administered to the patient in a dose of 150 mg BID on consecutive days throughout the course of treatment.
29. The method of any one of embodiments 23-28, wherein Compound 1 in the pharmaceutical composition has the solid form obtained from Example 5.
30. A method of inhibiting the production of inhibiting the production of 2-HG in a R132C mutated IDH-1 enzyme at no more than about 5 times the inhibition of 2-HG production in a R132H mutated IDH-1 enzyme, the method comprising contacting an IDH-1 enzyme not having arginine at position 132 with a composition comprising Compound 1 under conditions and for a time effective to inhibit 2-HG production in either an IDH-1 R132C or an IDH-1 R132H mutation in the mIDH-1 enzyme.
31. A method of treating a cancer in an adult patient, the cancer having a known mIDH-1 frequency of about 10-90%, the method comprising administering to a patient diagnosed with an IDH-1 mutation comprising an IDH-1 mutation selected from the group consisting of R132C, R132H, R132L, R132G, and R1325, the method comprising administering to the patient in need thereof a pharmaceutical composition comprising a total of 150 mg of a pharmaceutically acceptable solid form of 5-{[(1S)-1-(6-chloro-2-oxo-1,2-dihydroquinolin-3-yl)ethyl]amino}-1-methyl-6-oxo-1,6-dihydropyridine-2-carbonitrile, twice per day on consecutive days for a course of treatment comprising 6 months.
32. The method of embodiment 31, wherein the patient is diagnosed as having an IDH-1 mutation in a cell from the patient using a next-generation sequencing (NGS)-based tumor genotyping assay.
33. The method of any one of embodiments 31-32, wherein the pharmaceutical composition is administered to the patient every 12 hours.
34. The method of any one of embodiments 31-33, wherein Compound 1 in the pharmaceutical composition has the solid form obtained from Example 5.
35. A method of treating a chrondrosarcoma cancer having an IDH-1 mutation in an adult patient, the method comprising administering to the patient in need thereof a pharmaceutical composition comprising a total of 150 mg of a pharmaceutically acceptable solid form of 5-{[(1S)-1-(6-chloro-2-oxo-1,2-dihydroquinolin-3-yl)ethyl]amino}-1-methyl-6-oxo-1,6-dihydropyridine-2-carbonitrile, twice per day on consecutive days for a course of treatment comprising 6 months.
36. The method of embodiment 35, wherein the patient is diagnosed as having an IDH-1 mutation in a cell from the patient using a next-generation sequencing (NGS)-based tumor genotyping assay.
37. The method of any one of embodiments 35-36, wherein the pharmaceutical composition is administered to the patient every 12 hours.

### EXAMPLES

### Example 1: Compound 1 Potently and Selectively Inhibited 2-HG Production in IDH-1 R132H and IDH-1 R132C Mutant Enzymes in Biochemical Assays, Compared to Wild Type IDH-1 Enzyme and Mutant IDH-2 Enzymes

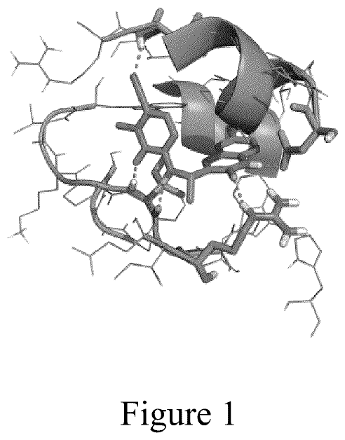

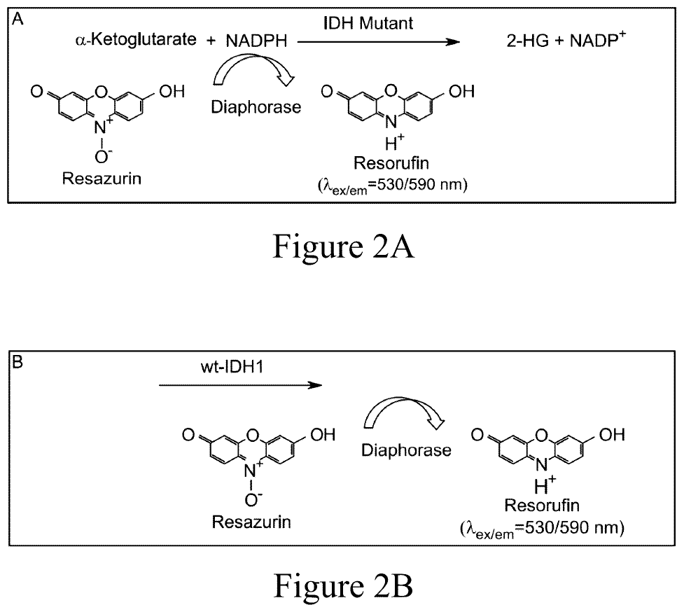

The biochemical potencies of Compound 1 against IDH-1 R132H and IDH-1 R132C mutants were determined in diaphorase-coupled assays, which measure activity by the determination of the level of remaining co-substrate NADPH after the enzymatic reaction is quenched (FIG. 1). FIG. 2A and FIG. 2B are schematics illustrating the working principle of the diaphorase-coupled assay for measuring potency and selectivity of Compound 1 for IDH-1 and IDH-2 enzymes. Recombinant homodimeric IDH-1 R132H or IDH-1 R132C mutant enzymes were used in these assays.

Results are shown in Table 2, relative to the IC₅₀ value obtained for R132H IDH-1 mutated enzyme. Referring to data in Table 2, Compound 1 was found to selectively inhibit the enzymatic activity of the IDH-1 R132H and IDH-1 R132C mutations with an IC₅₀ value within a factor of about 5 (i.e., the IC₅₀ value measured for IDH-1 R132C mutant enzyme was about 5 times higher than the IC₅₀ measured in the IDH-1 R132H mutated enzyme). The selectivity of Compound 1 against other IDH isozymes was also tested utilizing diaphorase coupled assays employing either wild-type IDH-1 or one of 2 alternate mutated forms of IDH-2, namely IDH-2 R172K and IDH-2 R140.

TABLE 2

| Target | Relative Enzymatic IC₅₀ (Average +/− SEM, nM) |
|---|---|
| IDH-1 R132H | 1.0 (±6.6%) |
| IDH-1 R132C | 5.1 (±6.1%) |
| Wild Type IDH-1 | 922 |
| IDH-2 R172K | >1,000 |
| IDH-2 R140Q | >4,000 (no activity measured) |

Compound 1 had comparatively very weak activity against wild type IDH-1 (IC₅₀ value of about 922 times greater than the IC₅₀ value measured for IDH-1 R132H). Compound 1 also demonstrated very weak activity against IDH-2 R172K that was more than 1,000 greater than the IC₅₀ value measured for IDH-1 R132H. Compound 1 did not show any inhibition of IDH-2 R140Q up to a concentration of 100 μM. These selectivity data indicate that Compound 1 is a potent and selective inhibitor of IDH-1 R132 mutations.

### Example 2: Compound 1 Exhibited Specific Binding to a Surface Containing Immobilized IDH-1 R132 Mutant Protein (Compared to a Comparator Surface With Immobilized BCL6), With Two Binding Sites Having Different Kd Values Detected by Surface Plasmon Resonance Analysis

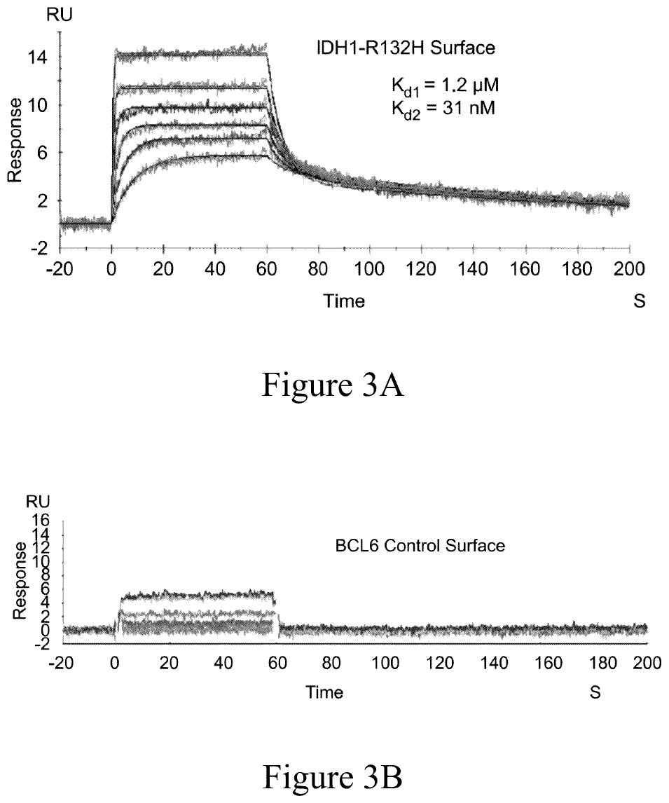

The biophysical interaction between Compound 1 and IDH-1 R132H was further characterized using Surface Plasmon Resonance (SPR) technology. Compound 1 was shown to exhibit specific binding to the surface containing immobilized IDH-1 R132H mutant protein compared to a control surface on which the unrelated protein BCL6 was immobilized, where no binding was observed (FIG. 3A and FIG. 3B, respectively). Analysis of the SPR data revealed two binding sites between Compound 1 and IDH-1 R132H, with Kd values of 31 nM (with kon1=2.04±0.03×105 M−1s−1 and koff1=0.0063±0.0001 s−1) and 1200 nM (with kon2=1.56±0.03×105 M−1s−1 and koff1=0.187±0.001 s−1), respectively. It is likely that the apparent low affinity binding site is an artifact of the immobilization of the protein on the surface of the chip, and as the Kd value for the high affinity binding site is close to the enzymatic IC₅₀ of Compound 1 for IDH-1 R132H, this was used to confirm specific binding of Compound 1 to IDH-1 R132H.

### Example 3: Compound 1 Potently Inhibited 2-HG Production in IDH-1 R132G, IDH-1 R132L, and IDH-1 R1325 Mutant Cell Lines in Cell Based Assays, With IC₅₀ Values Greater Than IDH-1 R132C Mutant Cell Lines

The cellular potency of Compound 1 in suppressing intracellular 2-HG levels was determined in cell lines expressing five different mutated IDH-1 proteins found in human cancers (R132H, R132C, R132G, R132L, R132S). The human fibrosarcoma cell line HT-1080 harbors a naturally occurring heterozygous IDH-1 R132C mutation. The human colorectal carcinoma cell line HCT 116 is wild type for IDH-1, but heterozygous mutations coding for IDH-1 R132H or R132C were introduced by knock-in into the endogenous IDH-1 gene locus. Finally, the human astrocytoma cell line U-87 MG is also wild type for IDH-1, but expression of five different mutated IDH-1 proteins was achieved by stable transfection.

The parental HCT116 line (colon) line does not produce high levels of 2-HG, but the variants used herein (X-MAN HCT-116 lines obtained from Horizon Discovery Ltd.) are engineered to knock-in a heterozygous mutation of either IDH-1 R132H or IDH-1 R132C. This recapitulates the cellular context in mIDH-1 cancer cells where there are both wild type and mutant IDH-1 subunits that together form a heterodimer that is responsible for the production of elevated levels of 2-HG. These modified lines can be used as models of IDH-1 mutant disease.

Each of these cell lines was treated with Compound 1 for 24 hr, and intracellular 2-HG levels were determined by mass spectroscopy. As shown in the Table 3, Compound 1 suppressed 2-HG production in each cell line, with IC₅₀ values ranging from less than 10 nM to less than 150 nM. Compound 1 is therefore a potent inhibitor of a variety of clinically relevant IDH-1 mutations in a cellular context. Table 3 shows the IC₅₀ values measured relative to the IC₅₀ value obtained for U-87 MG/IDH-1 R132G.

TABLE 3

| Cell Line | Relative 2-HG IC₅₀ (nM)* |
|---|---|
| U-87 MG/IDH-1 R132G | 1.0 (±30%) |
| U-87 MG/IDH-1 R132S | 1.17 (±21%) |
| U-87 MG/IDH-1 R132H | 1.29 (±17%) |
| U-87 MG/IDH-1 R132L | 5.39 (±22%) |
| U-87 MG/IDH-1 R132C | 7.00 (±30%) |
| HCT 116(IDH-1 R132H/+) | 3.36 (±19%) |
| HT-1080(IDH-1 R132C/+) | 9.66 (18%) |
| HCT 116(IDH-1 R132C/+) | 13.96 (±18%) |

*Mean +/− SEM where applicable

### Example 4: Testing Compound 1 in Mouse Xenograft Models Using HCT 116 Cells With R132 C and R132H Mutations

In order to optimize the dosing schedule of Compound 1 to achieve sustained >90% 2-HG inhibition in mIDH-1 in vivo, HCT116 (IDH-1 R132H)and HCT116 (IDH-1 R132C) xenograft-bearing mice were treated with Compound 1 at 25 and 50 mg/kg BID (3 doses). The free drug concentration of Compound 1 at 12 hour post final dose is above the in vivo IC₉₀ for all doses, and a greater than 90% reduction of 2-HG levels in tumor were achieved in each case. The free drug concentration decreased to 3-10× the in vivo IC₅₀ at 24 hour post final dose, and Compound 1 showed 80-90% inhibition. There was less than 20 nM free drug concentration in tumor at 48 and 72 hours after final dose, and at that point there was less than 50% 2-HG inhibition in tumor samples, consistent with the reduced level of Compound 1.

### Example 5: Pharmaceutical Compositions in an Oral Dosage Form of Compound 1

A therapeutically effective amount of Compound 1 can be orally administered (e.g., an amount providing a steady state blood concentration greater than the IC₉₀ for 2-HG production for cancer cells having the IDH-1 R132 mutation disclosed herein, and less than an amount of about 7,200 ng/mL). For example, a therapeutically effective amount of Compound 1 can provide a steady state blood concentration of about 2,000 ng/mL to 7,200 ng/mL throughout the course of treatment. The therapeutically effective amount can be up to about 150 mg of Compound 1 in the solid form obtained by the method of Example 5, administered to the patient BID on consecutive days throughout a course of treatment of at least about 6 months.

Step 1: Compound 1 can be obtained using the chemical synthesis disclosed in PCT patent application publication WO2016/044789A1 (published Mar. 24, 2016; filed Sep. 18, 2015). Examples 1, 21 and 25 from WO2016/044789A1 are incorporated herein by reference, along with associated analytical methods disclosed in the publication WO2016/044789A1. Briefly, Compound 1 can be obtained using the method of Example 25 (pages 92-93), based on the reaction of Intermediate II-1 (obtainable using the method of Example 1 on pages 26-27) and Intermediate III-1 (obtainable using the method of Example 21 on pages 79-82). Using this method, Compound 1 was obtained as a white solid (790 mg). m.p. 262-264° C. 1H NMR (300 MHz, DMSO-d₆) δ: 12.07 (s, 1H), 7.75 (s, 1H), 7.73 (d, J=2.2 Hz, 1H), 7.51 (dd, J=8.6, 2.3 Hz, 1H), 7.31 (d, J=8.8 Hz, 1H), 6.97 (d, J=8.0 Hz, 1H), 6.93 (d, J=7.7 Hz, 1H), 5.95 (d, J=8.0 Hz, 1H), 4.68 (m, 1H), 3.58 (s, 3H), 1.50 (d, J=6.6 Hz, 3H). LCMS (Method 3): 100% pure @ 254 nm, Rt 10.78 min, m z 355, 357 [M+H]+. The filtrate and the colored fractions (TLC pure) from the second ISCO were combined and treated with activated charcoal and filtered (until the filtrate is colorless). The filtrate was then concentrated under reduced pressure on rotavap to remove dichlorometane until a lot of white solid precipitated out. The white solid was collected by filtration and washed with cold MeOH. It was then mixed with MeCN/H₂O (10 mL/25 mL) and lyophilized to afford the title compound 1-13 as a white solid (970 mg). m.p. 262-264° C. 1H NMR (300 MHz, DMSO-d₆) δ: 12.06 (s, 1H), 7.75 (s, 1H), 7.73 (d, J=2.5 Hz, 1H), 7.51 (dd, J=8.6, 2.3 Hz, 1H), 7.31 (d, J=8.8 Hz, 1H), 6.97 (d, J=8.0 Hz, 1H), 6.92 (d, J=8.0 Hz, 1H), 5.95 (d, J=8.0 Hz, 1H), 4.68 (m, 1H), 3.58 (s, 3H), 1.50 (d, J=6.9 Hz, 3H). LCMS (Method 3): 100% pure @ 254 nm, m/z 355, 357 [M+H]⁺. The total yield for combined two batches is >67%.

Step 2: Next, a solid form of Compound 1 can be obtained that is useful in an oral dosage form. Unless otherwise indicated, the studies in Examples 1-4,6 and 7 were performed using a pharmaceutically acceptable solid form in an oral dosage form of Compound 1 that can be obtained by the method of Step 2 of Example 5. All volumes are with respect to the quantity of Compound 1 (v/w). Compound 1 obtained from Step 1 above is dissolved in 18 volumes of dichloromethane. The resulting solution is then concentrated under reduced pressure to approximately 5 volumes. To the mixture is added 5 volumes of ethyl acetate. The mixture is concentrated under reduced pressure to 5 volumes. To the mixture is added an additional 5 volumes of ethyl acetate, and the mixture again concentrated under reduced pressure to 5 volumes. The mixture is diluted to 10 volumes with ethyl acetate, and the mixture stirred at room temperature for 18 hours and then cooled to 0° C. The mixture is stirred at 0° C. for 3 hours and then filtered. The solids are rinsed with ethyl acetate and dried under vacuum (counterbalanced by nitrogen) at ambient temperature.

Step 3: The oral dosage form of Compound 1 is a pharmaceutically acceptable solid form of Compound 1, can be obtained using the method of Example 5 Step 2. The oral dosage form does not contain associated solvent or a counter ion. In particular, the oral dosage form of Compound 1 can be a capsule comprising drug substance (Compound 1) blended with excipients to improve powder flow and encapsulated in a Coni-Snap® hard gelatin capsule suitable for oral dosage in humans.

A pharmaceutically acceptable solid form of Compound 1 can be identified using reflection X-ray powder diffraction (XRPD) pattern of Compound 1. High resolution X-ray Powder Diffraction experiments can be performed with Panalytical X'Pert3 Powder XRPD on a Si zero-background holder. The 2 theta position can be calibrated against Panalytical 640 Si powder standard. Details of the XRPD method are listed below in Table 4, with XRPD peaks reported as diffraction angles at 2 theta, with d-spacing measured in angstroms.

TABLE 4
Parameters for Reflection Mode

| X-Ray Wavelength | Cu, kα, Kα1, (Å): 1.540598, Kα2 (Å): 1.544426 |
|---|---|
| | Kα2/Kα1 intensity ration: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.0131 |
| Scan speed (°/s | 0.033 |

An example of a pharmaceutically acceptable solid form of Compound 1 is a solid form characterized by a reflection X-ray powder diffraction (XRPD) pattern comprising characteristic peaks at 6.3, 12.8, 13.8, 23.6, and 27.8 degrees±0.2° 2θ. A pharmaceutically acceptable solid form of Compound 1 is a solid form characterized by characterized by an X-ray Powder Diffraction (XRPD), having diffractions at angles (2 theta±0.2) of 6.3, 12.8, 13.8, 23.6, and 27.8, corresponding to d-spacing (angstroms±0.2) of 14.0, 6.9, 6.4, 3.8, and 3.2, respectively. In some embodiments, a pharmaceutically acceptable solid form of Compound 1 can be identified by X-ray Powder Diffraction (XRPD), having characteristic diffractions at angles (2 theta±0.2) of 5.7, 6.3, 8.5, 10.6, 12.8, 13.8, 17.3, 22.0, 22.8, 23.6, and 27.8. In some embodiments, a pharmaceutically acceptable solid form of Compound 1 can be identified by X-ray Powder Diffraction (XRPD), having characteristic diffractions at angles (2 theta} 0.2) of 5.7, 6.3, 8.5, 10.6, 12.8, 13.8, 17.3, 22.0, 22.8, 23.6, and 27.8, corresponding to d-spacing (angstroms±0.2) of 15.4, 14.0, 8.4, 6.9, 6.4, 5.1, 4.0, 3.9, 3.8, and 3.2, respectively.

### Example 6: Comparative Compounds Demonstrated Greater Disparity Between 2-HG Inhibition in R132C and R132H IDH-1 Cells, Compared to Compound 1

The comparative activity of each of a series of mIDH-1 inhibitor compounds including Compound 1 were measured using the cell based assay in Example 3. The ratio of the IC₅₀ values obtained from IDH-1 R132C HCT116 mutant cells (IC₅₀ uM g mean)/IC₅₀ values obtained from IDH-1 R132H HCT116 mutant cells (IC₅₀ uM g mean) is provided in Table 5. Compound 1 had the lowest ratio among the tested compounds, indicating near equipotent activity of Compound 1 as measured with the R132C and R132H IDH-1 mutant cell assay of Example 3 (using the HCT 116 cells described in Example 3). Compound 1 showed comparative activity inhibiting 2-HG production from mIDH-1 R132C and R132H cell lines (using the assay of Example 3) that was within 5-fold, compared to more disparate differences in activity ranging from about 8-fold to over 200 fold (240) in comparative compound A-H in Table 5.

TABLE 5

| | | Ratio of IC₅₀ measured for |
|---|---|---|
| Compound | Structure | [IC₅₀ for R132C]/[IC₅₀ for R132H] |
| 1 | 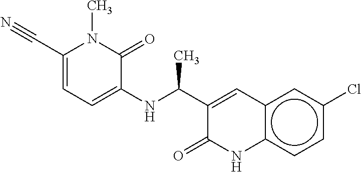 | 4.5 |
| A | 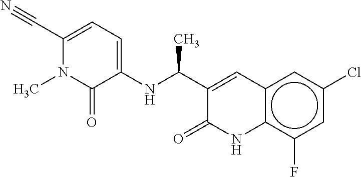 | 8.0 |
| B | 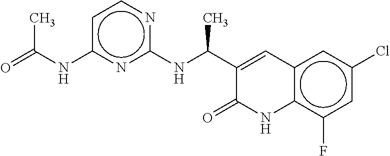 | 8.0 |
| C | 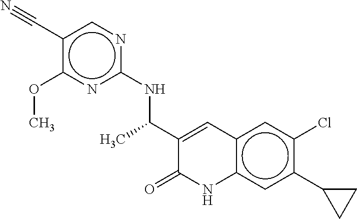 | 8.5 |
| D | 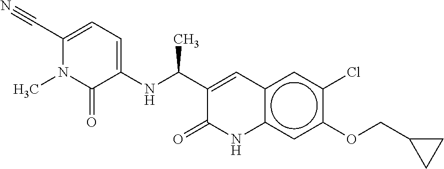 | 9.0 |
| E | 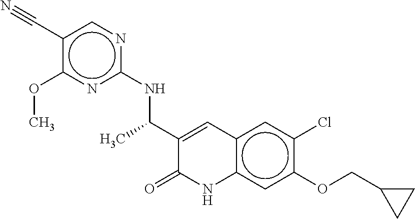 | 11.0 |
| F | 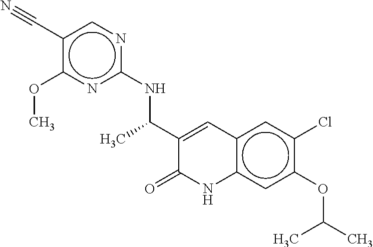 | 26 |
| G | 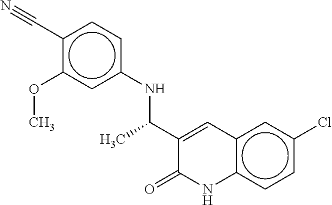 | 30 |
| H | 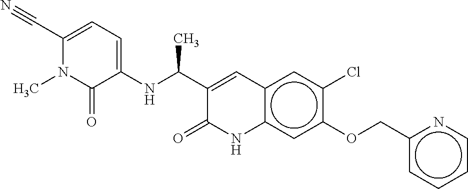 | 240 |

### Example 7: Compound 1 Induces Mutation Clearance in Patients with Acute Myeloid Leukemia (AML) or Myelodysplastic Syndrome (MDS) Treated in Phase 1 Dose Escalation and Expansion Study

Isocitrate dehydrogenase 1 mutations (mIDH-1) occur in 7-14% of AML patients (“pts.”) and 3% of MDS pts. Compound 1 is a highly potent, selective small molecule inhibitor of mIDH-1 without anticipated CYP or QTc liabilities at the recommended phase 2 dose.

This study evaluated the safety, pharmacokinetics (PK), pharmacodynamics (PD) and clinical activity of the novel anticancer drug Compound 1, administered to patients with relapsed or refractory acute myeloid leukemia (AML) or myelodysplastic syndrome (MDS). Compound 1 is a potent, selective, orally bioavailable, small-molecule inhibitor of mutated isocitrate dehydrogenase 1 (IDH1) and is intended for the treatment of patients harboring IDH1 mutations, in both hematologic and solid tumors.

The presence of mutations at codon 132 in IDH1 imparts a neomorphic activity to the enzyme, resulting in the production of the “oncometabolite” (R)-2-hydroxyglutarate (2-HG), which has pleotropic roles in tumorigenesis. Studies in genetically engineered mouse models and models derived from cancer patient samples support the hypothesis that mutated IDH1 produces 2-HG, the downstream effects of which cause epigenetic changes that consequently block the proper differentiation of progenitor cells and lead to cancer. These data support the therapeutic rationale that inhibition of mutated IDH1 will lower (R)-2-hydroxyglutarate (2-HG) levels and restore normal cellular differentiation.

### Inclusion Criteria

To be considered eligible to participate in this study, a patient met the inclusion criteria listed below:
1. Pathologically proven AML (except acute promyelocytic leukemia with the t(15;17) translocation) or intermediate risk, high risk or very high risk MDS as defined by the World Health Organization (WHO) criteria or Revised International Prognostic Scoring System (IPSS-R) harboring IDH1-R132 mutations, and one of the following based on enrollment stage or treatment cohort:
   a. Single Agent Phase 1 Cohorts including Dose-Escalation/ Dose-Expansion: AML/MDS either R/R to standard therapy, or for whom standard treatments are contraindicated
   b. Combination (Compound 1+azacitidine) Phase 1 Dose-Escalation/Dose-Expansion (patients must meet one of the following):
      i. Patients with AML that is either R/R to standard therapy, or for whom standard treatments are contraindicated
      ii. Patients with MDS that is either R/R to standard therapy, or are treatmentnaïve, who are eligible for azacitidine therapy
   c. Combination (Compound 1 +cytarabine) Phase 1 Dose-Escalation/Dose-Expansion Cohort: Patients≥60 years with treatment-naïve AML for whom standard treatments are contraindicated
   d. Phase 2 Cohort 1 (Single Agent) only: AML R/R to standard therapy
   e. Phase 2 Cohort 2 (Single Agent) only: AML in morphologic CR/CRi after prior therapy (+/−HSCT) with residual IDH1-R132 mutation 0.01%) detected in the bone marrow
   f. Phase 2 Cohort 3 (Single Agent) only: R/R AML/MDS that have been previously treated with IDH1 inhibitor therapy AND for whom standard treatments are contraindicated
   g. Phase 2 Cohort 4 (Compound 1+Azacitidine) only: Patients <60 years old with R/R AML/MDS with no prior hypomethylating agent therapy AND no prior IDH1 inhibitor therapy
   h. Phase 2 Cohort 5 (Compound 1+Azacitidine) only: R/R AML/MDS that have inadequately responded to or have progressed on prior treatment with a hypomethylating agent
   i. Phase 2 Cohort 6 (Compound 1+Azacitidine) only: R/R AML/MDS that have been previously treated with a single agent IDH1 inhibitor as their last therapy prior to study enrollment
   j. Phase 2 Cohort 7 (Single Agent) only: Treatment naive AML patients for whom standard treatments are contraindicated
   k. Phase 2 Cohort 8 (Compound 1+Azacitidine) only: Treatment naive AML patients who are candidates for azacitidine as a first line treatment
      (Note for Phase 2 Cohort 7 and Phase 2 Cohort 8: Treatment naive is defined as no prior treatment for AML. Patients may have received a prior treatment for another hematologic malignancy.)
2. Patients must have documented IDH1-R132 gene-mutated disease as evaluated by the site
3. Patients 18 years old
4. Eastern Cooperative Oncology Group (ECOG) performance status of 0 to 2
5. Signed informed consent prior to beginning study and undergoing procedures
6. No prior solid organ allograft
7. Acceptable liver function:
   a. Bilirubin 2 times upper limit of normal (ULN) (≤3 times ULN in patients with Gilbert Syndrome)
   b. Aspartate transaminase (AST, also referred to as SGOT), alanine transaminase (ALT, also referred to as SGPT) and alkaline phosphatase (ALP)≤3 times ULN
8. Acceptable renal function:
   a. Serum creatinine 1.5 times ULN or calculated creatinine clearance≥50 mL/min (Cockcroft and Gault 1976)
9. Recovery from the non-hematologic toxic effects of prior treatment to Grade≤1, or baseline value according to NCI CTCAE classification (excluding infertility, alopecia, or Grade 1 neuropathy)
10. Baseline QTcF 450 msec (average of the QTcF values of screening triplicate ECGs) Note: This criterion does not apply to patients with a bundle branch block (BBB); for patients with BBB, a cardiology consult is recommended to ensure that QTcF is not prolonged.
11. Negative serum pregnancy test if female of childbearing potential
12. For fertile men and women, agreement to use highly effective contraceptive methods for the duration of study participation and 90 days after the last dose of study medication
13. Agreement for male patients not to donate sperm and for female patients of childbearing potential not to donate ova during the study and for 90 days after the final dose of study drug
14. Phase 2 Cohorts 1-8 (SA and combination) only: Pre-treatment peripheral blood and bone marrow aspirate available for retrospective central confirmation of IDH1-R132 mutation is required. Note: Central confirmation of IDH1-R132 mutation is not required for study enrollment.

### Exclusion Criteria

To be eligible for entry into the study, the patient did not meet any of the exclusion criteria listed below:
1. Phase 1 Single Agent Dose-escalation/Dose-expansion Cohorts and Phase 2 Cohorts 1, 4, 5, 7 and 8 only: Patients who have been treated with an IDH1 targeted therapy are excluded
2. Phase 2 Single Agent Cohorts 1-3 and 7 only: Patients with IDH2 mutation detection at baseline or history of IDH2m inhibitor treatment are excluded
3. History of prior malignancy unless disease-free for 12 months or considered surgically cured; time from diagnosis (including concomitant diagnoses)
4. Patients with symptomatic central nervous system (CNS) metastases or other tumor location (such as spinal cord compression, other compressive mass, uncontrolled painful lesion, bone fracture, etc.) necessitating an urgent therapeutic intervention, palliative care, surgery or radiation therapy
5. Patients with previous allogeneic HSCT, if they meet any of the following criteria: <100 days from time of HSCT; active acute or chronic graft vs. host disease (GvHD); or receiving immunosuppressive therapy as treatment or prophylaxis against GvHD Note: Doses<20 mg methylprednisolone (or its equivalent) daily are not an exclusion criterion.
6. Treatment with radiation therapy, major surgery (requiring general anesthesia) within one month prior to study drug dosing
7. Treatment with chemotherapy or small molecule anticancer therapeutic within five half-lives of the agent or within 21 days if the half-life is unknown. Patients reenrolling in Cohort 6 after relapse/progression on Cohort 1 are exempt from this washout requirement (i.e. can continue Compound 1 treatment until re-enrollment) 8. Treatment with an anticancer therapeutic antibody less than four weeks before first dose of study drug
9. Treatment with other experimental therapies or participation in another clinical trial within a period of time that is less than the cycle length or within 21 days prior to starting study drug, whichever is shorter
10. Patients unable to swallow oral medications, or patients with gastrointestinal conditions (e.g., malabsorption, resection, etc.) deemed by the Investigator to jeopardize intestinal absorption
11. Congestive heart failure (New York Heart Association Class III or IV) or unstable angina pectoris;

previous history of myocardial infarction within one year prior to study entry, uncontrolled hypertension, or uncontrolled arrhythmias
12. Patients with a family history of QT prolongation
13. Concomitant medication(s) known to cause Torsades de Pointes (TdP) initiated less than the duration required to reach steady-state plasma concentration (approximately five half-lives) before first dose of study drug (medications used as needed [PRN] (e.g. Zofran) are exempt)
14. Concurrent treatment with chronic corticosteroids except if chronic treatment with <20 mg of methylprednisolone daily or equivalent (pulse steroids for treatment or prophylaxis are allowed [e.g., for transfusion or medication reactions])
15. Known HIV positivity
16. Active, uncontrolled bacterial, viral, or fungal infections, requiring systemic therapy (prophylactic systemic antimicrobials permitted)
17. Uncontrolled disease-related metabolic disorder (e.g., hypercalcemia)
18. Pregnant or nursing women or women of childbearing potential not using highly effective contraception; male patients not using highly effective contraception. Note: Women of childbearing potential and men must agree to use highly effective contraception prior to study entry and for the duration of study participation and 90 days after. Should a woman become pregnant or suspect she is pregnant while participating in this study, she should inform her treating physician immediately.
19. Serious nonmalignant disease (e.g., hydronephrosis, liver failure, or other conditions) that could compromise protocol objectives in the opinion of the Investigator and/or the Sponsor
20. Unwillingness or inability to comply with procedures either required in this protocol or considered standard of care
21. Medical, uncontrolled disease-related metabolic disorder, psychiatric, cognitive, or other conditions that may compromise the patient's ability to understand the patient information, give informed consent, comply with the study protocol, or complete the study
22. History of severe allergic reaction to azacitidine (if patient enrolling into azacitidine combination cohort) or low-dose cytarabine (if patient enrolling into cytarabine combination cohort)
23. Prisoners or patients who are involuntarily incarcerated or are compulsorily detained for treatment of either a psychiatric or physical (e.g. infectious disease) illness. Note: Under certain specific circumstances, a person who has been imprisoned may be included or permitted to continue as a patient, if local regulations permit. Strict conditions apply and FORMA's approval is required.

### Primary Outcome Measures

The outcome of the study can be evaluated using the following criteria:
1. Maximum Tolerated Doses (MTDs) or Maximum Evaluated Doses (MEDs) [Phase 1]. Time Frame: Within first 4 weeks of treatment.
2. Number of Participants with a Dose Limiting Toxicity (DLT) [Phase 1]. Time Frame: Within first 4 weeks of treatment. DLT Criteria can include:
   ≥GR 3 non-hematologic toxicity or laboratory finding
   Gr 4hematologic toxicity by Day 42 in absence of disease
   Inability to tolerate at least 75% of Cycle 1 treatment
3. Doses recommended for future studies [Phase 1]. Time Frame: Within first 4 weeks of treatment.
4. Complete Response (CR, CRi, MLFS, Marrow CR) Rate of Compound 1 as a single-agent or in combination with Azacitidine in patients with AML/MDS [Phase 2]. Time Frame: As per IWG Response Assessment Guidelines for AML and MDS based on investigator's assessment through study completion, e.g. modified IWG AML 2003/MDS 2006.

### Secondary Outcome Measures

The outcome of the study can also be evaluated using the following criteria:
1. Area under the plasma concentration versus time curve (AUC) [Phase 1 and Phase 2]. Time Frame: Blood samples for PK analysis collected at multiple visits during the first 60 days of treatment and on day 1 of all cycles following the first 30 days.
2. Peak Plasma Concentration (Cmax) [Phase 1 and Phase 2]. Time Frame: Blood samples for PK analysis collected at multiple visits during the first 60 days of treatment and on day 1 of all cycles following the first 30 days.
3. Time of peak plasma concentration (Tmax) [Phase 1 and Phase 2]. Time Frame: Blood samples for PK analysis collected at multiple visits during the first 60 days of treatment and on day 1 of all cycles following the first 30 days.
4. Time for half of the drug to be absent in blood stream following dose (T 1/2) [Phase 1 and Phase 2]. Time Frame: Blood samples for PK analysis collected at multiple visits during the first 60 days of treatment and on day 1 of all cycles following the first 30 days.
5. Rate at which drug is removed from blood stream (CL/F) [Phase 1 and Phase 2]. Time Frame: Blood samples for PK analysis collected at multiple visits during the first 60 days of treatment and on day 1 of all cycles following the first 30 days.
6. Rate of drug distribution within the blood stream (Vd/F) [Phase 1 and Phase 2]. Time Frame: Blood samples for PK analysis collected at multiple visits during the first 60 days of treatment and on day 1 of all cycles following the first 30 days.
7. Reduction of 2-HG levels in plasma [Phase 1 and Phase 2]. Time Frame: Blood samples for PK/PD analysis collected at multiple visits during the first 60 days of treatment and on day 1 of all cycles following the first 30 days.
8. Evidence of antileukemic or antimyelodysplastic activity of Compound 1 as determined by complete response (CR), CRi (complete remission with incomplete hematologic recovery), morphologic leukemia-free state (MLFS), Marrow CR, partial remission (PR), and stable disease (SD) as a single-agent or in combination with azacitidine or cytarabine [Phase 1]. Time Frame: As per IWG Response Assessment Guidelines for AML and MDS based on investigator's assessment through study completion.
9. Incidence and severity of adverse events, clinical laboratory abnormalities, and changes in ECG parameters as assessed by CTCAE v4.0 as a single-agent or in combination with azacitidine [Phase 2]. Time Frame: Safety will be assessed from time of first dose through 28 days post last dose.
10. Additional measures of antileukemic or antimyelodysplastic activity as determined by CRh, Overall Response (OR), and Stable Disease of Compound 1 alone or in combination with azacitidine [Phase 2]. Time Frame: As per IWG Response Assessment Guidelines for AML and MDS based on investigator's assessment through study completion.
11. Time to Response (TTR) [Phase 2]. Time Frame: From first dose of study drug through time of first response by blood recovery count.
12. Duration of Response (DOR) [Phase 2]. Time Frame: From time of first response by blood recovery count through relapse.
13. Event-Free Survival (EFS) [Phase 2]. Time Frame: From time of entry on study through progression.
14. Overall Survival (OS) [Phase 2]. Time Frame: From time of entry on study through death or date last known alive at end of follow-up.

### Disease History and Baseline Characteristics of Participants

A summary the disease history and participant demographics is provided below:

TABLE 6
Demographics and Disease History

| | | Compound 1 + |
|---|---|---|
| | Compound 1 | AZA |
| Characteristic | (n = 32)* | (n = 46) |
| Age, median (range), years | 72 (35-87) | 67 (31-88) |
| Female, % | 50 | 52 |
| ECOG PS-0/1/2. % | 28/50/22 | 28/57/15 |
| AML, n | 26 | 39 |
| Relapsed | 14 | 11 |
| >12 mo | 4 | 1 |
| ≤12 mo | 10 | 10 |
| Refractory | 8 | 15 |
| Treatment-naïve | 4 | 13 |
| Prior regimens, median (range)** | 2 (0-9) | 3 (0-6) |
| HMA (azacitidine/decitabine) | 12 | 9 |
| IDHm inhibitor | 1 | 4 |
| Investigational | 2 | 2 |
| HSTC | 2 | 3 |
| MDS, n | 6 | 7 |
| Relapsed/Refractory | 4 | 2 |
| Treatment-naïve | 2 | 5 |
| Prior regimens, median (range) | 1 (0-4) | 0 (0-4) |
| HMA (azacitidine/decitabine) | 4 | 2 |

*Including 3 pts treated with 100 mg QD with food.
**Not inclusive of all types; pt could have received more than one type

A summary of the baseline disease characteristics is shown below:

TABLE 7
Baseline Disease Characteristics

| | | | | |
|---|---|---|---|---|
| | R/R AML | TN AML | MDS** | All AML + MDS |
| All SA and CO | (n = 48) | (n = 17) | (n = 13) | (n = 78) |

IDH1 mutation type*, n

| | | | | |
|---|---|---|---|---|
| R132C | 23 | 10 | 5 | 38 |
| R132H | 13 | 3 | 6 | 22 |
| R132S | 6 | 2 | 0 | 8 |
| R132G | 5 | 2 | 1 | 8 |
| R132L | 1 | 0 | 0 | 1 |

Concurrent mutations*, n

| | | | | |
|---|---|---|---|---|
| FLT3 | 12 | 0 | 1 | 13 |
| NPM1 | 12 | 1 | 1 | 14 |
| CEBPA | 1 | 0 | 1 | 2 |
| TP53 | 3 | 0 | 1 | 4 |
| IDH2 | 1 | 1 | 0 | 2 |

*As reported by investigator per local tests
**One pt with R100 mutation

A summary of the Investigator-Assessed Response is shown below:

TABLE 8
Investigator-Assessed Response

| | Compound 1 SA | | Compound 1 + AZA | |
|---|---|---|---|---|
| | R/R AML | All Pts | R/R AML | All Pts* |
| Response | (n = 22) | (n = 32) | (n = 26) | (n = 45) |
| ORR, n (%)** | 9 (41) | 12 (38) | 12 (46) | 26 (58) |
| [95% Cl] | [21, 64] | [21, 56] | [27, 67] | [42, 72] |
| CR/CRm, n (%) | 4 (18) | 5 (16) | 3 (12) | 14 (31) |
| CRh, n (%) | 3 (14) | 3 (9) | 1 (4) | 1 (2) |
| Cri, n (%) | 2 (9) | 3 (9) | 6 (23) | 9 (20) |
| MLFS, n (%) | 0 | 0 | 2 (8) | 2 (4) |
| Marrow CR, n (%) | N/A | 1 (3) | N/A | 0 |
| SD, n (%) | 5 (23) | 9 (28) | 11 (42) | 14 (31) |
| PD, n (%) | 2 (9) | 3 (9) | 1 (4) | 1 (2) |
| NE, n (%) | 6 (27) | 8 (25) | 2 (8) | 4 (9) |

*one pt excluded from efficacy analysis due to the lack of a R132X mutation; pt continued on treatment and achieved a marrow CR
**ORR = overall response rate (CR/CRm + CRh + Cri + MLFS + Marrow CR)

### Variant Allele Frequency (VAF) Analysis

229 samples (213 from white blood cells (PaxGene and EDTA) and 16 from bone marrow analysis) were obtained from 59 AML patients treated with either Compound 1 as a single agent or Compound 1 in combination with azacitidine in the Phase 1 study. Next generation sequencing was carried out through target enrichment using HaloPlex® Target followed by Illumina® sequencing; coverage>100X across 75 genes. Digitial droplet PCR (ddPCR) was carried out through an input of 20 ng on a Stilla 3-channel system; VAF data based on >20,000 droplets.

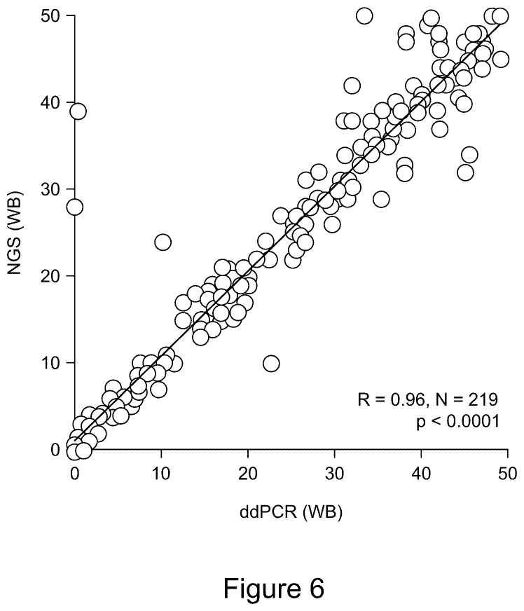

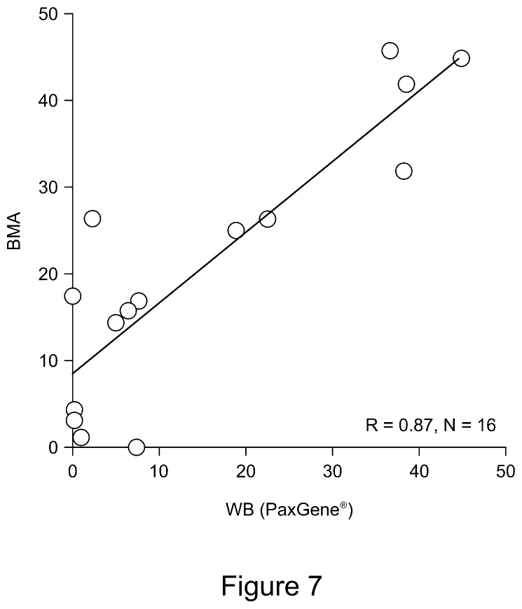

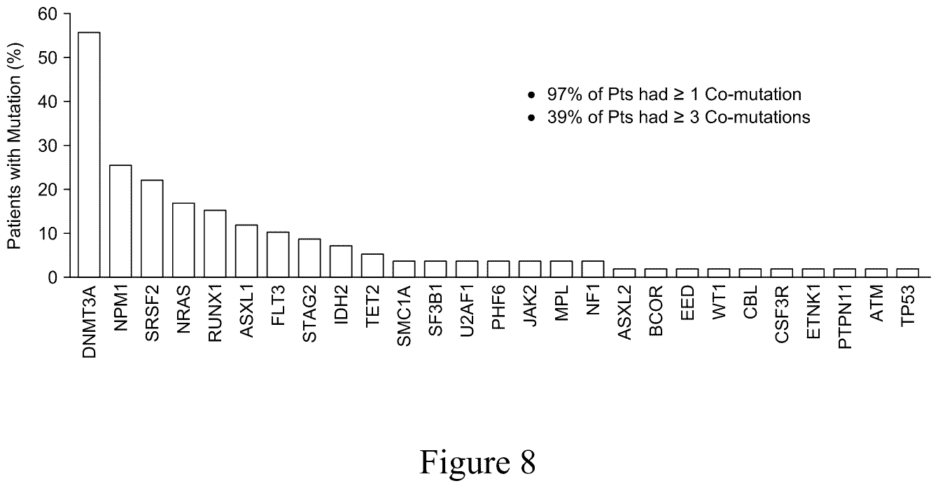

As shown in FIG. 6, good correlation between ddPCR and NGS was observed, which justifies using ddPCR for on-treatment assessment of IDH1 VAF. As shown in FIG. 7, detection of IDH1 from BMA can be useful in patients with low IDH1 VAF in WB.

Of the 59 pts with local and central IDH1m results (all sample types included), 53/59 (90%) central testing confirmed presence of IDH1m at study entry.

### Clinical Activity

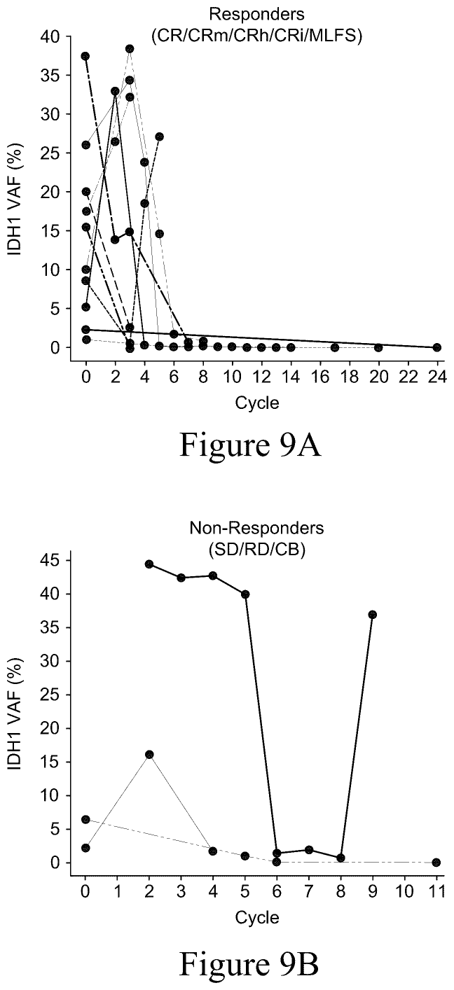

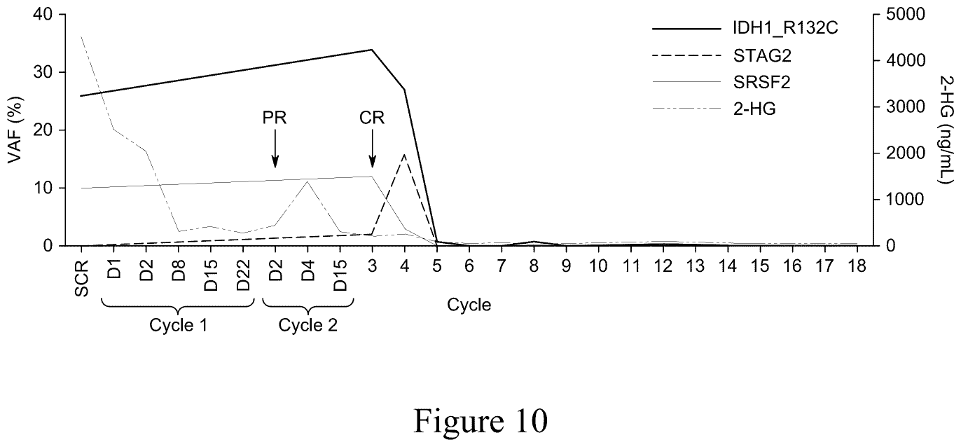

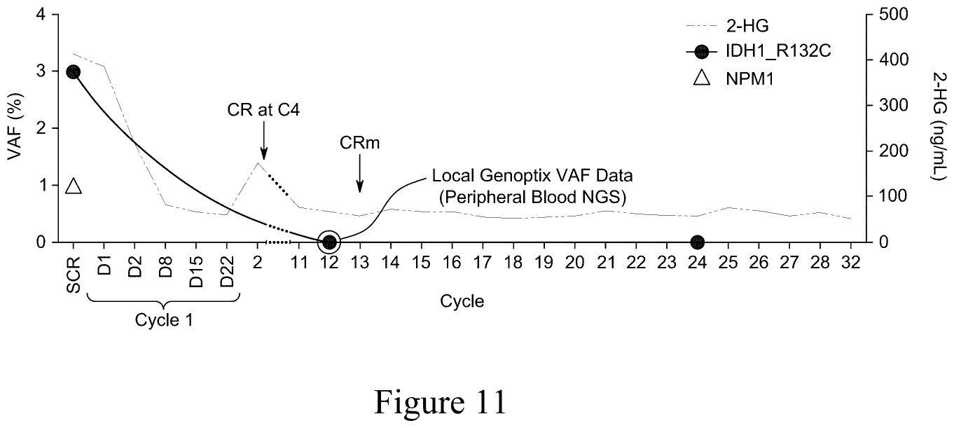

As shown in FIGS. 9A-12, upon treatment with Compound 1, significant reduction in IDH1 VAF across categories was observed. 25 patients achieved an objective response and 6 patients with SD had available longitudinal samples for analysis (VAF at C3). IDH1 mutation clearance/significant reduction is observed in 10/25 (40%) patients with an IWG response to Compound 1 (FIG. 9A). In patients with stable disease, 3/6 (50%) had clearance/significant reduction of the IDH1m VAF (FIG. 9B). FIGS. 10 and 11 show that clinical response is associated with a decrease in 2-HG and clearance of the IDH1m clone.

### Mechanism Resistance/Escape

The following details two case studies of individual patients.

### Case Study 1: IDH2-Mediated Resistance

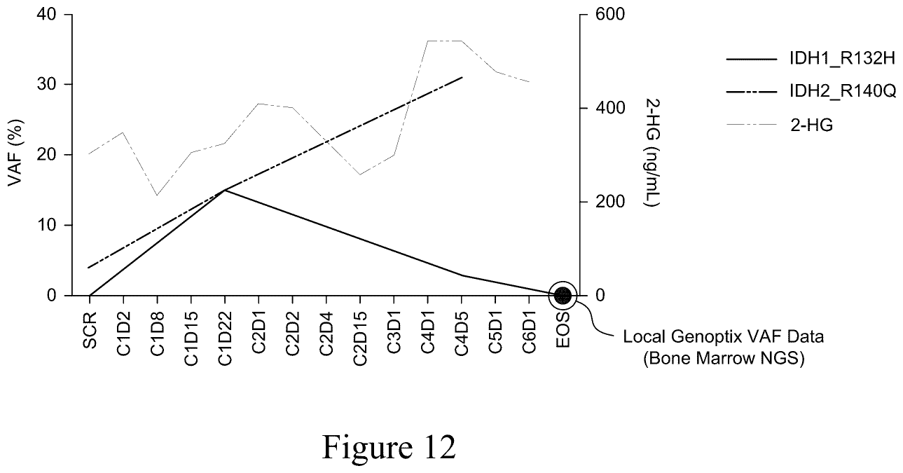

As shown in FIG. 12, an R/R AML patient with known IDH2m at baseline was treated with Compound 1 in combination with azacitidine. The patient remained in stable disease for 6 cycles then progressed. Compound 1 induced clearance of the IDH1m clone, however azacitidine was not effective in controlling the IDH2m clone that eventually drove the clinical progression.

### Case Study 2: Presence of Additional Non-IDH1m Clones Drive Resistance

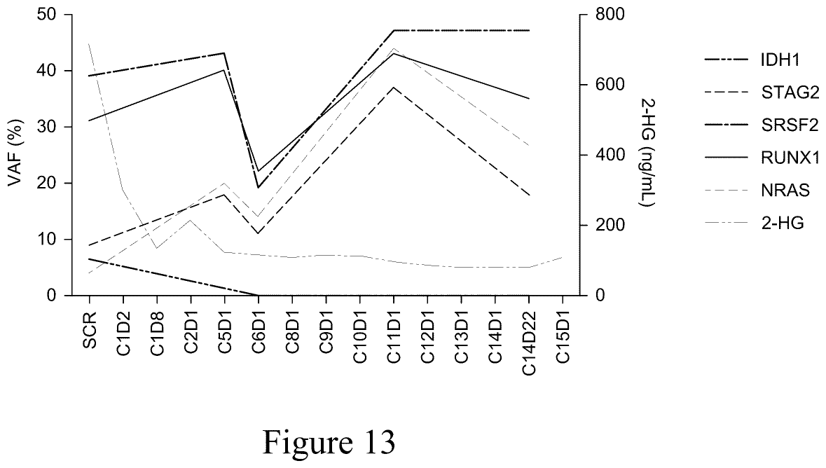

A treat naive AML secondary to MDS patient treated with Compound 1 as a single agent. As shown in FIG. 13, this patient remained stable for 15 cycles with no achievement of an IWG response, however IDH1 mutation clearance and normalization of 2-HG were observed.

### Conclusion

Compound 1 demonstrates clinical activity as a single agent and in combination with azacitidine in a high-risk Phase 1 population of AML/MDS patients with IDH1 mutation. In R/R AML, 41% and 46% pts achieve ORR with Compound 1 as a single agent and Compound 1 in combination with azacitidine treatment, respectively. 90% of pts enrolled with a history of IDH1m determined locally had a IDH1m confirmed centrally. Baseline co-mutation analyses demonstrated no correlation with clinical response (likely due to the small number of patients). Compound 1 induces IDH1 mutation clearance or significant reduction in treatment naive and R/R AM L patients regardless of IWG response. Of the 25 patient that achieved an objective response and with available samples (VAF at ≥C3), 10 (40%) had clearance or significant VAF reduction to <1%. Six stable disease patients had samples available and three (50%) had clearance or significant VAF reduction. Initial analysis of patients who relapse/progress on Compound 1 suggests non-IDHm-driven mechanism of escape.

### Example 8: Diagnostic for Identifying AML Patients Having a Susceptible mIDH1 Mutation

Abbott REALTIME IDH1 is a commercially available, FDA-Approved in vitro polymerase chain reaction (PCR) assay for the qualitative detection of single nucleotide variants (SNVs) coding five IDH1 R132 mutations (R132C, R132H, R132G, R132S, and R132L) in DNA extracted from human blood (EDTA) or bone marrow (EDTA). Abbott RealTime IDH1 is for use with the Abbott m2000rt System.

The Abbott RealTime IDH1 is indicated as an aid in identifying acute myeloid leukemia (AML) patients with an isocitrate dehydrogenase-1 (IDH1) mutation for treatment with an FDA-Approved mIDH1 inhibitor. This test is for prescription use only. The Abbott RealTime IDH1 detects single nucleotide variants (SNVs) coding five IDH1 mutations (R132C, R132H, R132G, R132S, and R132L) by using PCR technology with homogeneous real-time fluorescence detection. The assay uses human blood or bone marrow aspirate specimens and reports a qualitative result. The table below lists the IDH1 mutations detected by the Abbott RealTime IDH1 assay.

TABLE 9

| Codon | IDH1 Mutation | SNV |
|---|---|---|
| R132 | R132C | TGT |
| | R132H | CAT |
| | R132G | GGT |
| | R132S | AGT |
| | R132L | CTT |

1.-20. (canceled)

21. A method of treating a patient diagnosed with a form of cancer characterized by an IDH1 mutation selected from the group consisting of R132G, R132S and R132L, the method, comprising administering to the subject 300 mg of Compound 1 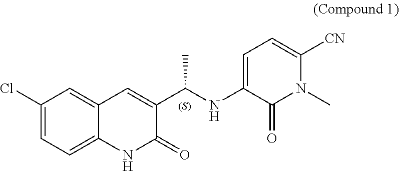
per day.

22. The method of claim 21, wherein the patient is diagnosed with a cancer characterized by a concurrent mutation selected from the group consisting of FLT3, NPM1, CEBPA and TP53.

23. The method of claim 21, wherein the patient is diagnosed with a cancer that is not characterized by an IDH2 mutation.

24. The method of claim 21, wherein the patient is diagnosed with acute myeloid leukemia (AML) or myelodysplastic syndrome (MDS) characterized by the IDH1 mutation.

25. The method of claim 24, wherein the patient is diagnosed with MDS or AML further characterized by a concurrent mutation selected from the group consisting of FLT3, NPM1, CEBPA and TP53.

26. The method of claim 21, wherein the patient is diagnosed with a cancer characterized by a concurrent mutation selected from the group consisting of DNMT3A, NPM1, SRSF2, NRAS, RUNX1, ASXL1, FLT3, STAG2, TET2, SMC1A, SF3B1, U2AF1, PHF6, JAK2, MPL, NF1, ASXL2, BCOR, EED, WT1, CBL, CSF3R, ETNK1, PTPN11, ATM and TP53.

27. A method of treating a patient diagnosed with a hematological malignancy characterized by an IDH1 mutation selected from the group consisting of R132C, R123H, R132G, R132S and R132L and a concurrent FLT3 mutation, the method comprising administering a total of 150 mg of Compound 1 orally twice per day (e.g. only twice per day) to the patient in need thereof:

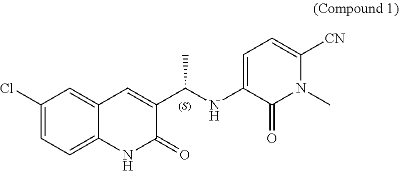

28. The method of claim 27, wherein the patient is diagnosed with a hematological malignancy characterized by a co-mutation selected from the group consisting of DNMT3A, NPM1, SRSF2, NRAS, RUNX1, ASXL1, STAG2, TET2, SMC1A, SF3B1, U2AF1, PHF6, JAK2, MPL, NF1, ASXL2, BCOR, EED, WT1, CBL, CSF3R, ETNK1, PTPN11, ATM and TP53.

29. The method of claim 27, comprising administering Compound 1 to the patient every day for 6 months.

30. The method of claim 27, wherein Compound 1 is administered to the patient as a single agent for the treatment of AML.

31. The method of claim 27, wherein Compound 1 is administered to the patient in combination with azacitidine during one or more 28-day treatment cycles, wherein
a. the azacitidine is administered to the patient at the dose of 75 mg/m² for 7 days IV/SC per every 28-day cycle; and
b. a total of 150 mg of Compound 1 is administered to the patient twice per day throughout the 28-day treatment cycles.

32. The method of claim 27, wherein the patient does not have an IDH-2 mutation.

33. A method of treating a patient diagnosed with a form of cancer characterized by an IDH1 mutation selected from the group consisting of R132G, R132S and R132L, the method comprising orally administering to the patient in need thereof a total amount of 150 mg of Compound 1 BID to the patient in need thereof: 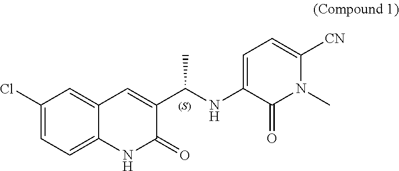
each day for a total of at least 6 months to treat the cancer characterized by the IDH1 mutation.

34. The method of claim 33, wherein the patient is diagnosed with a cancer characterized by a concurrent FLT3 mutation.

35. The method of claim 33, wherein the patient is diagnosed with a cancer characterized by a concurrent NPM1 mutation.

36. The method of claim 33, wherein the patient is diagnosed with a cancer characterized by a concurrent CEBPA mutation.

37. The method of claim 33, wherein the patient is diagnosed with a cancer characterized by a concurrent TP53 mutation.

38. The method of claim 33, wherein the patient is diagnosed with a cancer further characterized by both the mIDH1 mutation and a co-mutation selected from the group consisting of DNMT3A, NPM1, SRSF2, NRAS, RUNX1, ASXL1, STAG2, TET2, SMC1A, SF3B1, U2AF1, PHF6, JAK2, MPL, NF1, ASXL2, BCOR, EED, WT1, CBL, CSF3R, ETNK1, PTPN11, ATM and TP53.

39. The method of claim 34, wherein Compound 1 is administered as a single agent without azacitidine.

40. The method of claim 34, wherein Compound 1 is administered in combination with azacitidine to treat the mIDH1 cancer.
